(19) United States
(12) Patent Application Publication
(10) Pub. No.: US 2022/0151749 A1
(43) Pub. Date: May 19, 2022

(54) STEREOLITHOGRAPHICALLY PRODUCED SHAPED DENTAL PARTS AND METHOD FOR PRODUCTION FROM PHOTOPOLYMERIZABLE COMPOSITE RESIN COMPOSITIONS

(71) Applicant: Muhlbauer Technology GmbH, Hamburg (DE)

(72) Inventors: Jens Trager, Hetlingen (DE); Stephan Neffgen, Pinneberg (DE)

(21) Appl. No.: 17/436,361

(22) PCT Filed: Mar. 5, 2020
(86) PCT No.: PCT/EP2020/000056

§ 371 Date: Sep. 3, 2021

(30) Foreign Application Priority Data
Mar. 7, 2019 (DE) 10 2019 105 816.3

(51) Int. Cl. A61C 13/08; A61C 13/00; A61C 13/087; A61K 6/896; A61K 6/62; A61K 6/65; B33Y 10/00; B33Y 80/00; B33Y 70/00; B29C 64/124

(52) U.S. Cl. CPC A61C 13/082; A61C 13/0013; A61C 13/0019; A61C 13/087; A61K 6/896; B29K 2509/02; A61K 6/65; B33Y 10/00; B33Y 80/00; B33Y 70/00; B29C 64/124; A61K 6/62

(57) ABSTRACT

The invention relates to the use of flowable, photopolymerizable composite resin compositions that comprise a nanoscale organic surface-modified filler for stereolithographically producing a shaped dental part and to a method for producing corresponding shaped dental parts. The method according to the invention is particularly simple, fast, reliable and cost-effective. It allows improved shaped dental parts to be produced, in particular improved bridges and crowns.

The invention relates to a stereolithographic printing process (hereinafter also referred to as “3D printing process”) for producing shaped parts using a photopolymerizable composite resin composition.

The process of the invention is particularly simple, quick, reliable and inexpensive. It makes it possible to produce improved shaped parts, in particular improved dental prostheses such as bridges and crowns.

A known 3D printing process is, for example, bath-based photopolymerization such as stereolithography SLA and DLP. Here, the shaped parts are produced layer-by-layer under computer control (CAM) on the basis of a computer-aided design (CAD). Here, predetermined regions of thin layers of a liquid photopolymerizable composite resin are illuminated layer by layer, as a result of which polymerization, i.e. curing, takes place in each case in the illuminated region.

3D printing processes such as SLA and DLP require photopolymerizable resins which are sufficiently flowable for a polymerized layer to able to be coated very quickly and reliably with a next thin resin layer, in particular without such a resin layer having to be produced using an additional distribution element such as a doctor blade in the stereolithographic apparatus. Preferred resins therefore have a dynamic viscosity of less than about 5 Pa·s at room temperature (23° C.).

Difficulties associated with 3D printing processes are, in particular, the speed of printing, the dimensional accuracy in all three directions in space, the polymerization shrinkage, in particular in the stacking direction of the layers (z direction), the mechanical strength of the shaped parts and the color design and stability.

Depending on the use, the shaped parts have to meet various requirements. As dental prostheses, they have to meet particularly demanding requirements in respect of accuracy of fit, hardness, abrasion, strength, in particular bending strength and bending modulus, fracture toughness, tooth color and biocompatibility. Furthermore, the dental prosthesis should be able to be produced cheaply, particularly when it is used only as a temporary measure.

Dental prostheses composed of composite resin, in particular temporary crowns and bridges, are at present still produced by the dentist predominantly by a complicated process with the aid of tooth imprints, tooth (stump) models and polymerizable composite resins. A process of this type is known, for example, from EP1901676B1; corresponding materials are described, for example, in EP2034946B1, EP2070506B1, EP2198824B1 and EP2512400B1. The composite resins for temporary crowns and bridges have a pronounced yield point, i.e. in the rest state they virtually do not flow at all, while they flow when a shear stress is applied.

Especially in order to achieve the desired mechanical properties of crowns and bridges, temporary crown and bridge materials comprise not only free-radically polymerizable monomers, oligomers and polymers but usually also mixtures of, in particular, microfine inorganic fillers.

WO2005/084611 describes a filled, polymerizable dental material which contains a binder, a nanosize filler and a microfiller. The material can also be employed as temporary crown and bridge material. The dental materials of the examples contain from 70 to 85% by weight of filler particles. The nanosize filler is obtained by organic surface modification of commercially available agglomerated/aggregated nanofillers and is dispersed in a binder. It is assumed that the mixtures of binder and modified nanofiller obtained in this way are not suitable for general use as dental material since they have a high polymerization shrinkage and a low mechanical strength. These disadvantages are decreased only by mixing with microfillers. The dental materials of the examples are not flowable and not suitable for use in a 3D printer. The photopolymerized test specimens have bending strengths of up to 130 MPa.

WO2009/121337A2 describes a process for the stereolithographic production of shaped parts for medical purposes, in particular earmolds based on resin formulations, which are said to contain from 5 to 25% by weight of surface-modified nanoparticles, preferably from 5 to 15% by weight. The particles preferably have a particle size of <100 nm. The dispersions marketed by Clariant under the tradename Highlink are described as suitable particles. These are monodisperse SiO₂ sols in which all particles have about the same size. Such sols are complicated to produce and correspondingly expensive. In the examples, the resin formulations contained 9.6% by weight of silanized SiO₂. The bending strength was 135 MPa and the E modulus was 2810 MPa.

WO2013/153183 describes a process for the stereolithographic production of shaped dental parts, in particular dental components in the form of inlays, onlays, crowns and bridges based on composite resins. The composite resins are said to contain preferably from 40 to 90% by weight of fillers. The composite resins of the examples in each case contain more than 60% by weight of a filler mixture of pyrogenic silica, barium aluminum silicate glass powder and ytterbium fluoride in a weight ratio of 3:2:1. The composite resins have a viscosity significantly above 5 Pa·s. The photopolymerized test specimens have bending strengths of up to 84 MPa and a bending modulus of up to 2.5 GPa.

EP3040046A1 describes a process for the stereolithographic production of artificial teeth based on composite resins. The composite resins are said to contain preferably from 5 to 70% by weight of spherical fillers having average particle diameters of from 0.01 to 50 μm.

It is an object of the present invention to produce, by stereolithography, shaped dental parts, in particular crowns and bridges, inexpensively and with improved mechanical properties, or mechanical properties which are at least equivalent to conventional production processes. A further object is to keep the complication of technical apparatus when using the stereolithographic printing process as low as possible, in particular to dispense with a distributing element (doctor blade) for the composite resin composition used.

These objects are achieved according to the invention by the subject matter specified in the independent claims, with preferred embodiments of the invention being set forth in the dependent claims. In detail:

On the path to the present invention, the question arises of how to overcome the trade-off between
flowability of a photopolymerizable composite resin as basic prerequisite for use thereof in a stereolithographic process and
satisfactory mechanical strengths like the abovementioned requirements in respect of accuracy of fit, hardness, abrasion, strength, in particular bending strength and bending modulus, and fracture toughness.

This was, in particular, in the light of the abovementioned background according to which microfillers are added in addition to nanosize fillers to the composite resin composition to ensure the desired mechanical properties, in particular in the case of crowns and bridges. However, owing to this addition of microfillers, the flowability is impaired to such an extent that use in a stereolithographic process appears to be barely possible. This is especially the case when an additional distribution element (doctor blade) for the composite resin composition is dispensed with in the stereolithographic apparatus, i.e. the complication in terms of technical apparatus is to be kept low.

In the search for a solution to the abovementioned conflict of objectives and the disadvantages and problems associated with the abovementioned prior art, it was surprisingly and coincidentally found that the use according to the invention of nanodispersions as described in WO2005/084611 leads to shaped parts having equal or even improved properties, in particular mechanical properties, without other fillers being present, in particular without microfillers being present.

This was particularly surprising because microfillers have been said to have a significant importance for achieving the mechanical properties, in particular the desired bending modules and the bending strength, i.e. a person skilled in the art would therefore not have taken omission of these components into consideration. The applicant explains this phenomenon by the use of the specific surface-modified nanosize filler component (component “b” in the claims) being sufficient to ensure the desired mechanical properties of the dental material even in the absence of a microfiller component. This can ensure the flowability of the composite resin composition as fundamental prerequisite for use in a stereolithographic process, defined via the dynamic viscosity which, according to the claims, should be below 5 Pa·s at 23° C. In other words, the abovementioned object of the present invention has been achieved by a stereolithographic process (“3D printing process”, in particular an SLA or DLP process) using a photopolymerizable composite resin which has a viscosity of less than about 5 Pa·s, preferably less than about 3 Pa·s, and comprises the components as set forth in the claims.

The present invention accordingly provides, in particular, for the use of a flowable, photopolymerizable composite resin composition having a dynamic viscosity of less than 5 Pa·s at 23° C., preferably less than 3 Pa·s at 23° C., more preferably 0.5-2.5 Pa·s at 23° C., more preferably 1.0-2.0 Pa·s at 23° C., preferably measured using a plate-plate rheometer having an upper plate diameter of 25 mm at a shear stress of 50 Pa, comprising:

a) free-radically photopolymerizable monomers and/or oligomers, preferably mixtures of free-radically photopolymerizable monomers and oligomers,

b) an organically surface-modified and optionally partially agglomerated and/or aggregated nanosize filler incorporated in the composite resin composition, where
the primary particles of the filler have a primary particle size of less than 100 nm, preferably less than 80 nm, more preferably less than 60 nm, particularly preferably less than 40 nm, and
said filler in dispersion comprises dispersed primary filler particles and optionally filler aggregates and/or filler agglomerates, preferably at least 95% by volume, more preferably at least 98% by volume, more preferably at least 99% by volume, of said fillers present in dispersion comprise dispersed primary filler particles and optionally filler aggregates and filler agglomerates, having a diameter which is
   greater than 40 nm, preferably greater than 90 nm, and
   less than 1000 nm, preferably less than 800 nm, more preferably less than 600 nm, more preferably less than 400 nm, more preferably less than 200 nm, more preferably less than 150 nm,
and is, for example, in the range from 40 to 1000 nm, preferably from 40 to 800 nm, particularly preferably from 40 to 600 nm,

c) at least one photoinitiator,

d) optionally a stabilizer and

e) optionally pigment particles,

f) optionally a stabilized free radical

for the stereolithographic production of a shaped dental part, in particular bridges and crowns, based on said composite resin composition.

According to the invention, the nanosize filler according to feature b) is optionally still partially agglomerated and aggregated, i.e. some of the nanoparticles are agglomerated-aggregated particles in which two or more primary particles are joined by strong forces (aggregates) and these are partially joined to other aggregates by weak forces (agglomerates).

As a result, the complicated production of nanofillers consisting only of primary particles (for example by the sol-gel process) can be dispensed with and recourse can be made to, in particular, cheaper alternatives such as flame-pyrolytically produced silicon dioxide comprising nanosize primary particles which are held together both by strong aggregate forces (in particular sintering bonds) and weak agglomerate forces to form larger aggregates and/or agglomerates. The agglomerate bonds can be largely broken by mechanical incorporation of such fillers into the composite resin composition of the invention. The organic surface modification of the nanosize filler component b) according to the claims results in this being able to be dispersed in the composite resin and in renewed agglomeration of primary particles or aggregates/agglomerates to form larger associates with an increase in viscosity after incorporation into the composite resin not occurring. This organic surface modification can be, in particular, a silanization. The organic surface modification preferably introduces groups onto the surface of the nanosize fillers which can react chemically with the composite resin or have a high affinity for this composite resin.

The flowable, polymerizable composite composition preferably has, in a frequency sweep experiment in the range from 10⁻² Hz to 10⁻⁴ Hz, an intersection between the G′ curve and the G″ curve (G′=storage modulus, G″=loss modulus, in each case plotted as a function of the frequency), where G″>G′ at frequencies higher than the frequency at the intersection of the G′ curve with the G″ curve. The measurement is carried out at 23° C., for example using a plate-plate rheometer having an upper plate diameter of 25 mm at a gap of 0.1 mm and a deformation of 1% from 10 Hz to 10⁻⁴ Hz. For a definition, see Thomas G. Metzger, Das Rheologie Handbuch, 4th edition, Vincentz Network, 2012.

Said composite resin composition has an optimized optical density for the photopolymerization to be employed, in particular at wavelengths of 405 nm or 385 nm. This results, in particular, in achievement of a high dimensional accuracy of the shaped dental parts, and in particular reduces further curing in the z direction (z-overcuring).

Likewise, it was surprisingly found in storage experiments on the pigment-containing composite resin composition of the invention that the pigments incorporated for coloring do not sediment during storage over more than 3 months (and even more than 12 months). This was surprising because it would have to have been assumed that particles having an increasing size, in particular above the nanosize range (as in the case of the pigments used), would tend to sediment in composite resin compositions provided for 3D printing. For this reason, conventional pigmented composite resins also have to be homogenized before use in stereolithographic apparatuses in order to obtain an optimal result. However, storage-stable homogeneous composite resins could surprisingly be provided according to the invention. The composite resins displayed no sedimentation which would have had an adverse effect on mechanical properties or the tooth color of the printed shaped bodies after a storage time of 3, 6 or even 12 months. In particular, a photopolymerizable composite resin in which pigment microparticles also present do not sediment during storage was obtained according to the invention.

Improved shaped dental parts could be produced by 3D printing processes using, in particular, a photopolymerizable composite resin containing the following key components:

a) free-radically polymerizable monomers and/or oligomers, preferably mixtures of free-radically photopolymerizable monomers and oligomers,

b) an organically surface-modified and optionally partially agglomerated and/or aggregated nanosize filler incorporated in the composite resin composition, where
the primary particles of the filler have a primary particle size of less than 100 nm, preferably less than 80 nm, more preferably less than 60 nm, particularly preferably less than 40 nm, and
said filler in dispersion comprises dispersed primary filler particles and optionally filler aggregates and filler agglomerates, preferably at least 95% by volume, more preferably at least 98% by volume, more preferably at least 99% by volume, of said fillers present in dispersion comprise dispersed primary filler particles and optionally filler aggregates and/or filler agglomerates, having a diameter which is
   greater than 40 nm, preferably greater than 90 nm, and
   less than 1000 nm, preferably less than 800 nm, more preferably less than 600 nm, more preferably less than 400 nm, more preferably less than 200 nm, more preferably less than 150 nm,
and is, for example, in the range from 40 to 1000 nm, preferably from 40 to 800 nm, particularly preferably from 40 to 600 nm,

c) photoinitiator,

and the dynamic viscosity of the photopolymerizable composite resin at 23° C. is less than 5 Pa·s, preferably measured using a plate-plate rheometer having an upper plate diameter of 25 mm at a shear stress of 50 Pa.

The nanosize filler particles present in the composite resin composition of the invention can have a number of features which are summarized briefly below.

The particles b) consist essentially of aggregates of primary particles as are typically formed during production of pyrogenic silica. The size of the primary particles can, for example, be determined by transmission electron microscopy. The shape of the particles b) is essentially not ideally spherical but irregular, in particular in aggregates. The particles b) are present in a dispersion essentially as small agglomerates having a diameter of less than 1000 nm or in unagglomerated form. The particles b) have a heterodisperse size distribution.

The particles sizes in dispersion are distributed over a continuous particle size range from at least about 40 nm to not more than 1000 nm, preferably not more than 600 nm. The particle size distribution can be determined by means of various methods known to a person skilled in the art, for example by means of dynamic light scattering.

The average particle size diameter (z-average of the dynamic light scattering) of the filler agglomerates or aggregates and/or individual particles present in dispersion is preferably in the range from 90 to 500 nm, more preferably from 150 to 350 nm. The average particle size diameter (z-average of the dynamic light scattering) of the filler agglomerates or aggregates and/or individual particles present in dispersion can, for example, be determined by means of dynamic light scattering in 2-butanone or the mixture of the free-radically photopolymerizable monomers and/or oligomers (component a)). The term “z-average” refers to the measured average particle diameter weighted according to scattered light intensities. The individual particle sizes of the fillers present in dispersion (filler agglomerates or aggregates and unaggregated/unagglomerated filler particles) is determined from the measured data of the dynamic light scattering by means of the method described in the section “Measured values and methods”.

One possible way of quantitatively determining the particle size distribution even in the presence of large particles (e.g. pigments or microfillers) is an analytical system based on flow field-flow fractionation (flow FFF). Such a system can be obtained, for example, under the model designation “AF2000 AT” from Postnova Analytics GmbH, Landsberg, Germany. The separation range extends over a particle size range of 1 nm-100 μm. The fractionation of the sample under mild conditions according to particle size occurs, due to the different diffusion coefficients of differently sized particles in an open flow channel, without a stationary phase as is known to a person skilled in the art from, for example, HPLC or GPC. Suitable media are the solvents or dispersion media which have been mentioned above for the light scattering measurement. The measurement and evaluation software allows both the calculation of absolute particle sizes on the basis of the FFF theory and also based on a calibration with suitably sized particle size standards. Such particle size standards are obtainable, for example, under the name “NIST Traceable Size Standards” from Thermo Fisher Scientific, Fremont (Calif.), USA. A qualitative and also quantitative determination of the particle size distribution can be carried out by coupling of the fractionator with suitable detectors.

A separation of a dispersion into two particle size fractions can also be effected using a membrane having a suitable pore size. A gravimetric determination of amounts of particles which have been retained or have passed through the membrane is subsequently carried out. Suitable membranes are, for example, Teflon membranes having suitable pore sizes (e.g. membrane filters having a pore size of 1 μm as are obtainable in various sizes from Pieper Filter GmbH, Bad Zwischenahn, Germany under the name “PTFE auf Stützvlies, Typ TM”). The separation power of a particular membrane can be determined using the abovementioned size standards before analysis of a sample. Here, a standard having a size above the pore size of the membrane and a standard having a size below the pore size of the membrane is chosen and it is checked whether this is completely retained or passes completely through the membrane.

As mentioned at the outset, the nanosize filler incorporated into the composite resin composition, i.e. the component “b)” according to the invention, is organically surface-modified, as already described in principle in WO 2005/084611. Thus, the dispersed organically surface-modified nanosize and optionally partially agglomerated and/or aggregated nanosize filler particles can have been organically surface-treated before the dispersion process, preferably using a silane, or else not have been organically surface-treated and/or are surface-modified by the following steps:

i) provision of a composite resin composition by mixing said free-radically photopolymerizable monomers and/or oligomers as per the above-described component a) of the composite resin composition,

ii) addition of a silane hydrolysate to said mixture,

iii) dispersion of said nanosize filler particles as per component b), preferably pyrogenic silica, in said mixture,

where the ratio of silane hydrolysate to particle surface area of the agglomerated particles to be dispersed in step iii) is preferably in the range from 0.005 mmol/m² to 0.08 mmol/m² or from 0.01 mmol/m² to 0.02 mmol/m², in each case based on the molar amount of the silanes used per unit surface area of the filler.

A further suitable production process for the dispersed organically surface-modified nanosize and optionally partially agglomerated and/or aggregated nanosize filler particles, which can be used in the case of strongly surface-treated, preferably silanized, starting powders, comprises the steps:

i) provision of a composite resin composition by mixing said free-radically photopolymerizable monomers and/or oligomers as per the above-described component a) of the composite resin composition,

ii) dispersion of said surface-modified nanosize filler particles as per component b), preferably surface-modified pyrogenic silica, in said mixture.

This process can be particularly preferred in the case of silanized pyrogenic silica having an area-based carbon content of more than 4.5·10⁻⁴ g(carbon)/m²(filler surface area), preferably more than 7.0·10⁻⁴ g(carbon)/m²(filler surface area) and particularly preferably more than 12.0·10⁻⁴ g(carbon)/m²(filler surface area), where the filler surface area is to be determined by the BET method.

The agglomerated particles to be dispersed in step iii) or ii) preferably have a specific surface area determined by the BET method (in accordance with DIN 66131 or DIN ISO 9277) of less than 200 m²/g, preferably less than 100 m²/g and particularly preferably less than 60 m²/g. Suitable pyrogenic silicas are commercially available, for example Aerosil® 130, Aerosil® 90, Aerosil® Ox50 (in each case from Evonik Industries, Essen, Germany), HDK® S13, HDK® C10 and HDK® D05 (in each case from Wacker Chemie, Munich, Germany). In a further embodiment, the agglomerated particles to be dispersed are surface-treated before the dispersion process, for example with a silane. Agglomerated particles pretreated with a silane are, for example, Aerosil® R202, Aerosil® R805, Aerosil® R972 (Evonik Industries, Essen, Germany). In a particular embodiment, the agglomerated particles to be dispersed have been surface-modified before the dispersion process with a certain amount of silane which contains at least one free-radically polymerizable group. Suitable pyrogenic silicas which have been surface-modified in this way are obtainable, for example, under the name Aerosil® R7200 (Evonik Industries, Essen, Germany). The ratio of silanizing agent (step ii)) to particle surface area of the agglomerated particles to be dispersed in step iii) is preferably in the range from 0.005 mmol/m² to 0.08 mmol/m² or from 0.01 mmol/m² to 0.02 mmol/m².

Photopolymerizable composite resin compositions which are preferred according to the invention for use in a stereolithographic process (e.g. SLA, DLP) for the layer-by-layer buildup of a shaped dental part contain, based on 100% by weight of the total composition, the components a)-e) as follows:

a) 90-55% by weight, preferably 80-55% by weight, more preferably 75-60% by weight, of free-radically polymerizable monomers and/or oligomers, preferably mixtures of free-radically polymerizable monomers and oligomers,

b) 5-60% by weight, preferably 10-45% by weight, more preferably 20-45% by weight, more preferably 25-40% by weight, of an organically surface-modified and optionally partially agglomerated and/or aggregated nanosize filler incorporated into the composite resin composition, where
the primary particles of the filler have a primary particle size of less than 100 nm, preferably less than 80 nm, more preferably less than 60 nm, particularly preferably less than 40 nm, and
said filler in dispersion comprises dispersed primary filler particles and optionally filler aggregates and/or filler agglomerates, preferably at least 95% by volume, more preferably at least 98% by volume, more preferably at least 99% by volume, of said fillers present in dispersion comprising dispersed primary filler particles and optionally filler aggregates and/or filler agglomerates, having a diameter which is
   greater than 40 nm, preferably greater than 90 nm, and
   less than 1000 nm, preferably less than 800 nm, more preferably less than 600 nm, more preferably less than 400 nm, more preferably less than 200 nm, more preferably less than 150 nm,
and is, for example, in the range from 40 to 1000 nm, preferably from 40 to 800 nm, particularly preferably from 40 to 600 nm,

c) 0.01-5% by weight of photoinitiator,

d) 0.001-5% by weight of stabilizer,

e) 0-5% by weight, preferably 0.01-5% by weight, of pigment particles,

f) 0-5% by weight, preferably 0.0025-0.05% by weight, of stabilized free radical,

where the photopolymerizable composite resin contains at least 85% by weight, preferably at least 90% by weight, more preferably at least 95% by weight, of a) and b) in total.

Photopolymerizable composite resin compositions which are particularly preferred according to the invention for use in a stereolithographic process (e.g. SLA, DLP) for the layer-by-layer buildup of a shaped dental part contain, based on 100% by weight of the total composition, the components a)-e) as follows:

a) 75-60% by weight of free-radically polymerizable (meth)acrylates,

b) 25-40% by weight of silanized nanosize filler particles having particle sizes (z-average of dynamic light scattering) of the individual particles and/or filler agglomerates and/or filler aggregates present in dispersion preferably in the range from 90 to 500 nm, more preferably from 150 to 350 nm,

c) 0.1-2% by weight of photoinitiator,

d) 0.1-1% by weight of stabilizer,

e) 0.01-1% by weight of pigments,

where the photopolymerizable composite resin contains at least from 96 to 99.89% by weight of a) and b) in total.

The invention further provides a process for producing a shaped dental part, in particular a bridge and crown, comprising the steps:

i) provision of a flowable, photopolymerizable composite resin composition having a dynamic viscosity of less than 5 Pa·s at 23° C., preferably less than 3 Pa·s at 23° C., more preferably 0.5-2.5 Pa·s at 23° C., more preferably 1.0-2.0 Pa·s at 23° C., preferably measured using a plate-plate rheometer having an upper plate diameter of 25 mm at a shear stress of 50 Pa, comprising the components a)-c) and optionally the components d) and e) as described above for the composite resin compositions, and

ii) stereolithographic layer-by-layer buildup of the dental material from the flowable, photopolymerizable composite resin composition in a bath filled with said composite resin composition.

The invention further provides a shaped dental part, in particular a bridge or crown, as is obtainable by this above-described process. The shaped dental part obtained in this way preferably has a bending strength of at least 100 MPa, preferably at least 130 MPa, and/or a bending modulus of at least 3 GPa, preferably at least 4 GPa, measured in accordance with ISO 4049:2009. Apart from the bridges and crowns mentioned, further parts used for prosthetic, conserving and preventative dentistry come into consideration as shaped dental parts. Without making any claim of completeness, some representative examples of use may be mentioned: dental fillings, inlays, onlays, stump buildups, artificial teeth and facings.

Suitable components a), b), c), d), e) and f) of the photopolymerizable composite resin composition according to the present invention are known to a person skilled in the art from the prior art. For the sake of completeness, these will be described by way of example below.

### Component a)

The component a) comprising free-radically polymerizable monomers and/or oligomers and preferably mixtures of monomers and oligomers has a dynamic viscosity at 23° C. of 0.05-5 Pa·s, preferably 0.1-3 Pa·s, preferably measured using a plate-plate rheometer having an upper plate diameter of 25 mm at a shear stress of 50 Pa. As an alternative, the viscosity can also be measured using a coaxial cylinder system C25 as described in DIN 53019. Preferred monomers, oligomers and polymers are acrylates and methacrylates, more preferably mixtures of these. Suitable monomers and oligomers are monomers and oligomers selected from among methyl, ethyl, 2-hydroxyethyl, butyl, benzyl, tetrahydrofurfuryl or isobornyl (meth)acrylate, p-cumylphenoxyethylene glycol methacrylate, bisphenol A di(meth)acrylate, bis-GMA, ethoxylated or propoxylated bisphenol A dimethacrylate (e.g. SR-348c (Sartomer)) having 3 ethoxy groups or 2,2-bis[4-(2-methacryloxypropoxy)phenyl]propane, urethane dimethacrylate UDMA (an addition product of 2-hydroxyethyl methacrylate and 2,2,4-trimethylhexamethylene diisocyanate), diethylene, triethylene or tetraethylene glycol di(meth)acrylate, trimethylolpropane tri(meth)acrylate, pentaerythritol tetra(meth)acrylate and also glyceryl dimethacrylate and trimethacrylate, 1,4-butanediol di(meth)acrylate, 1,10-decanediol di(meth)acrylate or 1,12-dodecanediol di(meth)acrylate, 1,6-hexanediol dimethacrylate, tricyclo[5.2.1.0^{2,6}]decanedimethanol diacrylate, tricyclo[5.2.1.0^{2,6}]decanedimethanol dimethacrylate. Preferred (meth)acrylate monomers are benzyl, tetrahydrofurfuryl or isobornyl methacrylate, p-cumylphenoxyethylene glycol methacrylate, 2,2-bis[4-(2-methacryloxypropoxy)phenyl]propane, bis-GMA, UDMA, SR-348c. It is also possible to use N-monosubstituted or N-disubstituted acrylamides such as N-ethylacrylamide or N,N-dimethacrylamide or bisacrylamides such as N,N′-diethyl-1,3-bis(acrylamido)propane, 1,3-bis(methacrylamido)propane, 1,4-bis(acrylamido)butane or 1,4-bis(acryloyl)piperazine as free-radically polymerizable monomers. Preference is given to using mixtures of the abovementioned monomers.

### Component b)

Suitable particles for producing the particles b) are pyrogenic metal oxides, semimetal oxides or mixed metal oxides. Preference is given to pyrogenic silicon dioxide (pyrogenic silica) or pyrogenic mixed oxides of silicon, preferably pyrogenic mixed oxides of silicon with aluminum, zirconium and/or zinc.

Suitable silanes for the surface modification of the particles of component b) correspond to the following general formula

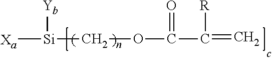

where R is a hydrogen atom or an alkyl group, X is a hydrolysable group (for example Cl or OCH₃), Y is a hydrocarbon radical, n is an integer from 1 to about 20, a is an integer from 1 to 3, b is 0, 1 or 2 and c is an integer in the range from 1 to 3 and a+b+c=4.

### Component c)

The photoinitiators which can be used here are characterized in that they can effect curing of the material by absorption of light in the wavelength range from 300 nm to 700 nm, preferably from 350 nm to 600 nm and particularly preferably from 380 nm to 500 nm, and optionally by additional reaction with one or more coinitiators. Suitable photoinitiators are, in particular, phosphine oxides, benzoins, benzil ketals, acetophenones, benzophenones, thioxanthones and mixtures thereof. Acylphosphine oxides and bisacylphosphine oxides such as 2,4,6-trimethylbenzoyldiphenylphosphine oxide or bis(2,4,6-trimethylbenzoyl)phenylphosphine oxide are particularly suitable. As a possible second photopolymerization initiator, use can be made of, in particular, diketones, acylgermanium compounds, metallocenes and mixtures thereof.

### Component d)

Suitable stabilizers are, in particular, benzotriazoles, triazines, benzophenones, cyanoacrylates, salicylic acid derivatives, hindered amine light stabilizers (HALS) and mixtures thereof. Particularly suitable stabilizers are o-hydroxyphenylbenzotriazoles such as 2-(2H-benzotriazol-2-yl)-4-methylphenol, 2-(5-chloro-2H-benzotriazol-2-yl)-4-methyl-6-tert-butylphenol, 2-(5-chloro-2H-benzotriazol-2-yl)-4,6-di-tert-butylphenol, 2-(2H-benzotriazol-2-yl)-4,6-di-tert-pentylphenol, 2-(2H-benzotriazol-2-yl)-4-methyl-6-dodecylphenol, 2-(2H-benzotriazol-2-yl)-4,6-bis(1-methyl-1-phenylethyl)phenol, 2-(2H-benzotriazol-2-yl)-6-(1-methyl-1-phenylethyl)-4-(1,1,3,3-tetramethylbutyl)phenol, 2-(2H-benzotriazol-2-yl)-4-(1,1,3,3-tetramethylbutyl)phenol or 3-(2H-benzotriazol-2-yl)-5-tert-butyl-4-hydroxybenzene propanoate, o-hydroxyphenyltriazines such as 2-(2-hydroxy-4-hexyloxyphenyl)-4,6-biphenyl, 1,3,5-triazine or 2-(2-hydroxy-4-[2-hydroxy-3-dodecyloxypropyloxy]phenyl)-4,6-bis(2,4-dimethylphenyl)-1,3,5-triazine, o-hydroxybenzophenones such as 2-hydroxy-4-octyloxybenzophenone, cyanoacrylates such as ethyl 2-cyano-3,3-diphenylacrylate, 2-ethylhexyl 2-cyano-3,3-diphenylacrylate or tetrakis[(2-cyano-3,3-diphenylacryloyl)oxymethyl]methane, hindered amine light stabilizers (HALS) such as N,N′-bisformyl-N,N′-bis(2,2,6,6-tetramethyl-4-piperidinyl)hexa-methylenediamine, bis(2,2,6,6-tetramethyl-4-piperidyl) sebacate, bis(1,2,2,6,6-pentamethyl-4-piperidyl) sebacate or methyl 1,2,2,6,6-pentamethyl-4-piperidyl-sebacate, salicylic esters and mixtures thereof.

### Component e)

Preferred pigments are, for example, the pigments marketed under the tradename Sicovit. Preferred pigments have particle sizes D50 in the range from 1 to 20 μm.

### Component f)

Suitable stabilized free radicals are, in particular, free radicals such as 2,2,6,6-tetramethylpiperidinyloxyl (TEMPO) and particularly preferably bis(2,2,6,6-tetramethyl-4-piperidyl-1-oxyl) sebacate. Stabilized free radicals are particularly preferably present in the photopolymerizable composite resin composition in an amount of 0.005-0.01% by weight.

### Further Components

In addition to the components a), b), c), d), e) and f), the photopolymerizable composite resin composition can contain further additives, in particular additives customary in dentistry, for example fluorescent dyes.

At this juncture, it may once again expressly be pointed out that the resolution of the conflict of objectives mentioned at the outset (flowability of a photopolymerizable composite resin as fundamental prerequisite for its use in a stereolithographic process versus satisfactory mechanical properties such as sufficiently high bending strength and bending modulus) has surprisingly been able to be achieved by largely dispensing with the microfillers originally considered to be responsible for the good mechanical properties when a composite resin composition as per the present patent application as defined in the claims is used.

In the light of this background, the photopolymerizable composite resin contains essentially no microfillers in a preferred embodiment of the invention. Or the maximum proportions of such fillers are 5% by weight, 1% by weight or preferably 0.5% by weight. For the purposes of the present invention, said microfillers are, in particular, milled fillers or spherical fillers having particle sizes in the range from 1 to 50 μm; these have characteristic particle shapes which differ significantly from those of the aggregated particles of component b) of the composite resin composition according to the invention. Furthermore, the photopolymerizable composite resin composition preferably does not contain any thixotropy-inducing agents, in particular (agglomerated) pyrogenic silica, i.e. pyrogenic silica which has not been surface-modified according to the process described. If a thixotropy-inducing agent is present, the proportions thereof should preferably be not more than 0.5% by weight, more preferably not more than 0.01% by weight.

If the pigments of the component e) or further additives have particle sizes of more than 1000 nm, the photopolymerizable composite resin contains less than 10% by weight, preferably less than 5% by weight and particularly preferably less than 1.0% by weight, of particles having particle sizes of more than 1000 nm.

The invention will be illustrated below with the aid of working examples. Firstly, an explanation will be given of various measurement and test methods used, as have already been comprehensively described in the prior art, for example WO 2005/084611; an example according to the invention follows subsequently.

### I. Measured Values and Methods

### 1. Particle Size and Particle Size Distribution

The particle size of the nanoparticles was determined by means of dynamic light scattering. A Zetasizer Nano ZS from Malvern Instruments Ltd. was used for this purpose. The measurement of the backscattered laser light was carried out in a backscattering arrangement at an angle of 175° to the optical axis of the laser. The evaluation of the information obtained from the correlator was carried out by the Zetasizer software on a PC. As analysis model, “General purpose (normal resolution)” was selected. The nanodispersion produced according to the invention was diluted with the resin mixture used in the particular case or with 2-butanone to a solids concentration of about 0.5% by weight, based on the amount of silica used. Measurement of the dilutions in resin mixture was carried out in disposable cells made of PMMA (polymethyl methacrylate) having a path length of 10 mm (LABSOLUTE®, from Th. Geyer GmbH & Co. KG, catalogue No. 7697102). Measurement of the dilutions in 2-butanone was carried out in fused silica cells having a path length of 10 mm (110QS, from Hellma).

### 2. Dynamic Viscosity (Shear Rates)

The dynamic viscosity was measured by means of a Kinexus DSR from Malvern Instruments Ltd. Here, a plate-plate geometry having a diameter of the upper plate of 25 mm was used. The measurement was carried out over a shear stress range from 1 Pa to 50 Pa. The value at a shear stress of 50 Pa was employed for the evaluation. The measurement is carried out at a constant sample temperature of 23° C., which was monitored and kept constant by the internal temperature control of the instrument.

### 3. Bending Strength and Bending Modulus

Bending strength and bending modulus were determined by a method analogous to ISO 4049:2009. For this purpose, rods having dimensions of 40 mm×2 mm×2 mm were printed with their longitudinal axis in the x or y direction of the construction space flat onto the building platform (the x and y axes span the plane in which the building platform lies, or parallel thereto the bottom of the tank, the z axis runs perpendicular to the x axis and the y axis). After cleaning away adhering resin residues with ethanol, the test specimens were illuminated again (Heraflash, from Heraeus Kulzer). The additional illumination was carried out for 180 s and after turning the test specimens through 180° around the longitudinal axis for a further 180 s.

Before measurement of the bending strength, the test specimens were stored in water at 37° C. for 24 hours. The measurement is carried out in a universal tester Z 010 or Z2.5 from Zwick at constant speed of advance of 0.8 mm/min until fracture occurred. The bending device used for this purpose consists of two steel rollers having a diameter of 2 mm which are applied parallel at a spacing of the axes of 20 mm and a third roller having a diameter of 2 mm which is mounted in the middle between the two others and parallel to them, so that the three rollers together can be used for three-point loading of the test specimen.

The calculation of the bending strength a and the bending modulus E is carried out by the measurement software according to the formulae

$$σ = \frac{3 Fl}{2 b {h}^{2}} and E = \frac{{l}^{3} F}{4 f b {h}^{3}}$$

F maximum force in newtons exerted on the test specimen

f deflection of the test specimen at a strain of 0.25%

I distance between the support points in mm

b width of the test specimen before the test in mm

h height of the test specimen before the test in mm

### 4. Three-Media Abrasion

The three-media abrasion was carried out on a Willytec three-media abrasion machine. For the relative assessment, test specimens were printed from the 3D printing material according to the invention and after-treated as described above under “Bending strength”. Test specimens composed of a conventional crown and bridge material from the cartridge (Luxatemp Automix Plus, from DMG) served as reference. These specimens were produced by curing of the automatically mixed pastes in a suitable metal mold. All test specimens were stored in water at 37° C. for 24 hours before the measurement. The test specimens were adhesively bonded onto the specimen wheel using a chemically curing cement and the gaps between the test specimens were filled up with a fluid, light-curing composite. The wheel was subsequently ground. The measurement was carried out over 50 000 cycles at a contact load of 15 N. The left-hand motor was set to a speed of rotation of 130 min⁻¹ and the right-hand motor was set to 60 min⁻¹. 150 g of milled millet which had been mixed with 220 g of distilled water to give a slurry served as abrasion medium.

After the end of the abrasion process, the test wheel was thoroughly rinsed under running water and dried using cellulose and compressed air. The profilometric measurement of the test specimens on the wheel was subsequently carried out (Profilometer Willytec DMA MESS V 1.12).

### 5. z-Overcuring

To determine the z-overcuring, a cuboidal test specimen having the following dimensions was printed: width about 50 mm, height about 25 mm, thickness about 5 mm. In the digital model of the test specimen, circular holes having diameters of 10 mm, 8 mm, 5 mm, 2.5 mm and 1 mm are provided. The test specimen is printed so that the areal vector of the planes of the circles lies orthogonal to the z axis (the x and y axes span the plane in which the building platform lies, or parallel thereto the bottom of the tank, the z axis runs perpendicular to the x axis and the y axis). After printing of the test specimen and cleaning as described above under “Bending strength”, the diameter of the holes is measured by means of a sliding caliper. A number of measurements parallel to the z axis (based on the printing process) and perpendicular thereto are carried out. An average is in each case formed from the two groups of measured values of a hole diameter. The diameter parallel to the z axis is subtracted from the diameter perpendicular thereto. The value obtained in μm is the z-overcuring.

### 6. Fracture Toughness (K_{1c} Value)

The method for determining the fracture toughness is based on the preliminary standard DIN CEN/TS 14425-5:2004 “Method for bending specimens with V notch (SEVNB method)”. The test specimens for determining the K_{1c} value are rods having the dimensions 50 mm×4 mm×3 mm (length×height×width). The production of the test specimens was, except for the different dimensions, carried out exactly as described above under “Bending strength and bending modulus”.

The test specimens are likewise stored in water at 37° C. for 24 hours before the measurement. They were subsequently marked in the middle of the 3 mm wide side using a pen and clamped into a holder. In this holder, the test specimens were notched to a notch depth of 1.0±0.2 mm using a slotted razor blade. In order to obtain a very sharp notch angle, the last cuts were made using an unused razor blade. The notch angle obtained here is about 30°.

The notched test specimens are loaded to fracture in a 4-point loading device at a constant speed of advance of 0.025 mm/min in a universal tester Z 010 or Z2.5 from Zwick. For the 4-point bending test, the supports have a spacing of 40.0 mm (±0.5 mm) and a radius of 5.0 mm (±0.2 mm). The load bearings have a spacing of 20.0 mm (±0.52 mm) and a radius of 5.0 mm (±0.2 mm). A uniform stress in the bending test is ensured by a gimbal arrangement. The load bearings are centered and arranged parallel over the supports.

Numbering of the test specimens ensures that the measurement result from the universal tester can later be assigned unambiguously to a particular test specimen.

The microscopic examination of the fracture surfaces is subsequently carried out. For each broken test specimen, one half is examined. This is shortened to such an extent that it can be positioned under an optical microscope with the fracture surface in the direction of the objective. An objective with 2.5-fold enlargement is selected. The further evaluation is carried out software-assisted with the aid of digital micrographs which are taken by a digital camera positioned on the microscope. The notch depth is measured at three places for each fracture surface examined and an average is formed therefrom. The average notch depth a of a test specimen should be in the range from 0.8 mm to 1.2 mm. The relative notch depth α of a test specimen is the ratio of average notch depth and thickness of the test specimen. This value should be in the range from 0.2 to 0.3. The stress intensity shape factor Y and the fracture toughness K_{1c} can then also be calculated therefrom. The K_{1c} is reported in the unit MPa m^{1/2}.

$$a = \frac{{a}_{1} + {a}_{2} + {a}_{3}}{3} \\ α = \frac{a}{W} \\ Y = 1.9887 - 1.326 α - \frac{( 3.49 - 0.68 α + 1.35 {α}^{2} ) α ( 1 - α )}{{( 1 + α )}^{2}} \\ {K}_{1 c} = \frac{F}{B \sqrt{W}} \frac{{S}_{1} - {S}_{2}}{W} \frac{3 \sqrt{α}}{2 {( 1 - α )}^{1.5}} Y$$

F maximum force exerted on the test specimen in MN

B width of the test specimen in m

thickness of the test specimen in m

S₁ spacing of the supports in m

S₂ spacing of the load bearings in m

a the average notch depth in m

a₁, a₂, a₃ measured notch depths in m

α the relative notch depth

Y stress intensity shape factor

Test specimens which have an average notch depth or a relative notch depth outside the intended values are disregarded. Likewise, test specimens which have inhomogeneities such as air bubbles are disregarded.

### 7. Frequency Sweep Test

The frequency sweep was carried out on a Kinexus DSR from Malvern Instruments Ltd. Here, a plate-plate geometry having a diameter of the upper plate of 25 mm was used. The sample was measured oscillating at frequencies of from 10 Hz to 0.0001 Hz at a gap of 0.1 mm and a deformation of 1%. 5 measuring points per decade were recorded. The measurement is carried out at a constant sample temperature of 23° C., which was monitored and kept constant by the internal temperature control of the instrument. The complex shear modulus G*, the storage modulus G′ (real part of the complex shear modulus), the loss modulus G″ (imaginary part of the complex shear modulus) and the loss factor tan δ (ratio of G″ and G′), inter alia, were recorded.

### 8. Production of the Particle Dispersions

A Dispermat® from VMA-Getzmann GmbH, model AE04-C1 was used for producing the particle dispersions. A toothed disk which had a diameter (D) of 70 mm and 12 teeth arranged approximately at right angles alternately on the two sides of the plane of the disk was used together with a double-wall stainless steel stirred vessel having an internal diameter of about 100 mm and a capacity of about 1 l. Furthermore, a toothed disk which had a diameter (D) of 90 mm and likewise 12 teeth arranged approximately at right angles alternately on the two sides of the plane of the disk was used together with a double-wall stainless steel stirred vessel having an internal diameter of about 180 mm and a capacity of about 5 l.

It was ensured that the internal diameter of the stirred vessel is from 1.3 D to 3 D and the distance of the main plane of the high-speed stirrer disk from the bottom of the stirred vessel is from 0.25 D to 0.5 D, where D is the diameter of the high-speed stirrer disk.

### II. Illustrative Composition

### Components Used

| | |
|---|---|
| Urethane | Genomer 4297, from Rahn AG |
| dimethacrylate | |
| Bisphenol A diglycidyl | X950-0000, from Esschem (CAS 1565-94-2) |
| ether methacrylate | |
| Triethylene glycol | Luvomaxx ® TEDMA, from Lehmann & Voss |
| dimethacrylate | & Co. KG (CAS 109-16-0) |
| Isobornyl methacrylate | SR423D, from Sartomer Europe division of |
| | Arkema (CAS 7534-94-3) |
| Hexanediol | X887 7446, from Esschem (CAS 6606-59-3) |
| dimethacrylate | |
| BHT | 2,6-Di-tert-butyl-4-methylphenol, from Merck |
| | (CAS 128-37-0) |
| Tinuvin 622 SF | From BTC Europe GmbH (CAS 65447-77-0) |
| 2,2,6,6-Tetramethyl- | T2324, from TCI Deutschland GmbH |
| 4-piperidyl | (CAS 31582-45-3) |
| methacrylate | |
| TPO | 2,4,6-Trimethylbenzoyldiphenylphosphine oxide, |
| | Omnirad TPO, from IGM Resins B.V. |
| | (CAS 75980-60-8) |
| Dynasilan ® Memo | 3-Trimethoxysilylpropyl methacrylate, from |
| | Evonik Resource Efficiency GmbH |
| | (CAS 2530-85-0) |
| Acetic acid | From Merck (CAS 64-19-7) |
| Deionized water | CAS 7732-18-5 |
| Aerosil ® Ox50 | From Evonik Resource Efficiency GmbH |
| | (CAS 112 945-52-5) |
| Admafine ® SO-C1 | Admatechs Company Limited |

A resin, i.e. a mixture of free-radically polymerizable monomers and oligomers, was produced. The monomers and oligomers were mixed until a homogenous solution was obtained. The resin had the following composition:

| | |
|---|---|
| Urethane dimethacrylate | 85 parts by weight |
| Bisphenol A diglycidyl ether methacrylate | 24 parts by weight |
| Triethylene glycol dimethacrylate | 40 parts by weight |
| Isobornyl methacrylate | 23 parts by weight |
| Hexanediol dimethacrylate | 28 parts by weight |

A silane hydrolysate was produced by adding 1.4 parts by weight of acetic acid and 10.6 parts by weight of water to 100 parts by weight of Dynasilan® MEMO.

The resin mixture (see table above) was firstly stirred at a low speed of rotation (300-500 min⁻¹).

9 parts by weight of the silane hydrolysate were added to 100 parts by weight of the resin and mixed into the resin mixture at a speed of rotation of 400 min⁻¹ for about one minute.

55 parts by weight of Aerosil® Ox50 were subsequently added a little at a time to the resin. The speed of rotation of the high-speed stirrer disk was varied from 1000 min⁻¹ to 1800 min⁻¹. During addition of a portion of the Aerosil® Ox50, the speed of rotation was briefly decreased to not less than 600 min⁻¹ in order to prevent severe dusting of the Aerosil. The addition procedure extended over a period of 1.5 h. The mixture was subsequently dispersed at a speed of rotation of 2000 min⁻¹ for 2 hours 15 minutes. A temperature in the range from 35° C. to 37° C. was established during this time. Overall, the total duration of the procedure was 4 hours.

A photopolymerizable composite resin, illustrative compositions 1, see table, was produced. For this purpose, initiators, stabilizers and pigments were added to the dispersion and the mixture was homogenized again for a few minutes. The photopolymerizable composite resin obtained had the following composition:

| Constituent [% by weight] | Example 1 |
|---|---|
| Dispersion | 98.18 |
| Color paste* | 0.14 |
| BHT (2,6-di-tert-butyl-4-methylphenol) | 0.08 |
| Tinuvin 622 SF | 0.2 |
| 2,2,6,6-Tetramethy1-4-piperidyl methacrylate | 0.2 |
| TPO | 1.2 |

*The color paste was a homogeneous mixture of 50% by weight of Sicovit pigment particles and a resin mixture composed of urethane dimethacrylate and triethylene glycol dimethacrylate.

The photopolymerizable composite resin had a dynamic viscosity of 1.4 Pa·s and was therefore most suitable for stereolithographic use.

The test specimens had the following mechanical properties:
The bending strength was 127±8 MPa (n=6/6).
The bending modulus was 4.69±0.17 GPa (n=6/6).
The three-media abrasion was −103.1±1.1 pm (n=6/6) (Luxatemp Automix Plus: −120.8±3.2 μm (n=6/6)).
The z-overcuring was 123 μm.
Furthermore, the shaped bodies have a comparatively improved fracture toughness (K_{1c} value: 0.88±0.12 MPa m^{1/2}), Vickers hardness (34.8±0.8 HV 0.3 (n=5/5)) and long-term color stability (ΔE 1.99 (28 d, 60° C.)).

### 3D Printing Process

Dental crowns and bridges were produced by means of a DLP printer (D 20 II from Rapid Shape GmbH Generative Production Systems) using the composite resin produced in this way. “DMG Luxaprint Crown” was selected as material in the Slicing Software Autodesk Netfabb Standard 2019.

The clinical fitting of the crowns and bridges was very good.

### COMPARATIVE EXAMPLE

Admafine® SO-C1 was used in the comparative experiment. This consists of spherical, essentially unaggregated particles. According to the manufacturer, these particles have an average particle size diameter of from about 200 to 400 nm and a specific surface area of from about 10 to 20 m²/g.

### Procedure

100 parts by weight of Admafine® SO-C1 were silanized using 3.8 parts by weight (about 0.01 mmol/m²) of Dynasilan® MEMO (silane hydrolysate) in a solvent mixture composed of 150 parts by weight of water and 300 parts by weight of methoxypropanol, as described in U.S. Pat. No. 6,890,968B2, page 8, subsequently dried and homogenized in a mortar.

A dispersion was then produced in a manner corresponding to the example according to the invention. For this purpose, 55 parts by weight of the methacrylate-silanized Admafine® SO-C1 were dispersed a little at a time in 109 parts by weight of the resin, which unlike the example according to the invention did not contain any silane hydrate.

After only 16 hours after the end of dispersing, separate phases had formed. While a small proportion by mass of the particles still formed a dispersion, the main part of the particles already formed a solid sediment. A storage-stable homogeneous dispersion had not been obtained.

1. The use of a flowable, photopolymerizable composite resin composition having a dynamic viscosity of less than 5 Pa·s at 23° C., preferably less than 3 Pa·s at 23° C., more preferably 0.5-2.5 Pa·s at 23° C., more preferably 1.0-2.0 Pa·s at 23° C., preferably measured using a plate-plate rheometer having an upper plate diameter of 25 mm at a shear stress of 50 Pa, comprising:
a) free-radically photopolymerizable monomers and/or oligomers, preferably mixtures of free-radically photopolymerizable monomers and oligomers,
b) an organically surface-modified and optionally partially agglomerated and/or aggregated nanosize filler incorporated into the composite resin composition, where
the primary particles of the filler have a primary particle size of less than 100 nm, preferably less than 80 nm, more preferably less than 60 nm, particularly preferably less than 40 nm, and
said filler in dispersion comprises dispersed primary filler particles and optionally filler aggregates and/or filler agglomerates having a diameter which is
greater than 40 nm, preferably greater than 90 nm, and
less than 1000 nm, preferably less than 800 nm, more preferably less than 600 nm, more preferably less than 400 nm, more preferably less than 200 nm, more preferably less than 150 nm,
and is, for example, in the range from 40 to 1000 nm, preferably from 40 to 800 nm, particularly preferably from 40 to 600 nm,
c) at least one photoinitiator,
d) optionally a stabilizer and
e) optionally pigment particles,
f) optionally a stabilized free radical
for the stereolithographic production of a shaped dental part, in particular bridges and crowns, based on said composite resin composition.

2. The use as claimed in claim 1, characterized in that the organically surface-modified nanosize filler and optionally partially agglomerated and/or aggregated nanosize filler particles to be dispersed have been surface-modified by the following steps:
i) provision of a composite resin composition by mixing said free-radically photopolymerizable monomers and/or oligomers as per component a) of the composite resin composition,
ii) addition of a silane hydrolysate to said mixture,
iii) dispersion of said nanosize filler particles as per component b), preferably pyrogenic silica, in said mixture,
where the ratio of silane hydrolysate to particle surface area of the agglomerated particles to be dispersed in step iii) is preferably in the range from 0.005 mmol/m² to 0.08 mmol/m² or from 0.01 mmol/m² to 0.02 mmol/m², in each case based on the molar amount of the silanes used per unit surface area of the filler.

3. The use as claimed in claim 1 or 2, characterized in that the nanosize filler particles to be incorporated into the composite resin composition have a specific surface area determined by the BET method of less than 200 m²/g, preferably less than 100 m²/g and particularly preferably less than 60 m²/g, and include pyrogenic silicas, for example Aerosil® 130, Aerosil® 90, Aerosil® Ox50, Aerosil® R7200, HDK® S13, HDK® C10 and HDK® D05.

4. The use as claimed in any of claims 1-3, characterized in that the composite resin composition comprises, based on 100% by weight of the total composition, the components a)-e) as follows:
a) 90-55% by weight, preferably 80-55% by weight, more preferably 75-60% by weight, of free-radically polymerizable monomers and/or oligomers, preferably mixtures of free-radically polymerizable monomers and oligomers,
b) 5-60% by weight, preferably 10-45% by weight, more preferably 20-45% by weight, more preferably 25-40% by weight, of an organically surface-modified and optionally partially agglomerated and/or aggregated nanosize filler incorporated into the composite resin composition, where
the primary particles of the filler have a primary particle size of less than 100 nm, preferably less than 80 nm, more preferably less than 60 nm, particularly preferably less than 40 nm, and
said filler in dispersion comprises dispersed primary filler particles and optionally filler aggregates and/or filler agglomerates having a diameter which is
greater than 40 nm, preferably greater than 90 nm, and
less than 1000 nm, preferably less than 800 nm, more preferably less than 600 nm, more preferably less than 400 nm, more preferably less than 200 nm, more preferably less than 150 nm,
and is, for example, in the range from 40 to 1000 nm, preferably from 40 to 800 nm, particularly preferably from 40 to 600 nm,
c) 0.01-5% by weight of photoinitiator,
d) 0.001-5% by weight of stabilizer,
e) 0-5% by weight, preferably 0.01-5% by weight, of pigment particles,
f) 0-5% by weight, preferably 0.0025-0.05% by weight, of stabilized free radical,
where the photopolymerizable composite resin contains at least 85% by weight, preferably at least 90% by weight, more preferably at least 95% by weight, of a) and b) in total,
and preferably
a) 75-60% by weight of free-radically polymerizable (meth)acrylates,
b) 25-40% by weight of silanized nanosize filler particles having particle sizes of the individual particles and/or filler agglomerates and/or filler aggregates present in the dispersion in the range from 90 to 500 nm, with an average particle size (z-average of dynamic light scattering) in the range from 150 to 350 nm,
c) 0.1-2% by weight of photoinitiator,
d) 0.001-5% by weight of stabilizer,
e) 0.01-1% by weight of pigments,
where the photopolymerizable composite resin contains at least from 96 to 99.89% by weight of a) and b) in total.

5. The use as claimed in any of claims 1-4, characterized in that the composite resin composition comprises pigments and has a storage stability over at least 3 months, preferably at least 6 months, more preferably over at least 12 months, without sedimentation of said pigments in the composite resin composition.

6. A process for producing a shaped dental part, in particular a bridge and crown, comprising the steps:
i) provision of a flowable, photopolymerizable composite resin composition having a dynamic viscosity of less than 5 Pa·s at 23° C., preferably less than 3 Pa·s at 23° C., more preferably 0.5-2.5 Pa·s at 23° C., more preferably 1.0-2.0 Pa·s at 23° C., preferably measured using a plate-plate rheometer having an upper plate diameter of 25 mm at a shear stress of 50 Pa, comprising the components a)-c) and optionally the components d) and e) as claimed in any of the preceding claims, and
ii) stereolithographic layer-by-layer buildup of the shaped dental part from the flowable, photopolymerizable composite resin composition in a bath filled with said composite resin composition.

7. A shaped dental part, in particular bridges and crowns, obtainable by the process as claimed in claim 6, wherein the shaped dental part preferably has
a bending strength of at least 100 MPa, preferably at least 130 MPa, and/or
a bending modulus of at least 3 GPa, preferably at least 4 GPa, measured in accordance with ISO 4049:2009.

8. The use as claimed in any of claims 1-5, the process as claimed in claim 6 or the shaped dental part as claimed in claim 7, characterized in that the nanosize filler has at least one feature selected from among the following:
it consists essentially of aggregates of primary particles as are formed in the production of pyrogenic silica,
the shape of the nanosize filler particles is essentially not ideally spherical but irregular, in particular in aggregates;
the nanosize filler particles are present in dispersion essentially as small agglomerates having a diameter of less than 1000 nm or in unagglomerated and/or unaggregated form;
the particles in dispersion are distributed over a continuous size range from at least about 40 nm to not more than 1000 nm, preferably not more than 600 nm;
the average particle size diameter, measured as z-average of dynamic light scattering, of the nanosize filler particles comprising filler agglomerates and/or filler aggregates and/or
unagglomerated/unaggregated filler particles present in dispersion is in the range from 90 to 500 nm, preferably from 150 to 350 nm.

9. The use as claimed in any of claims 1-5, the process as claimed in claim 6 or the shaped dental part as claimed in either of claims 7-8, characterized in that the composite resin composition comprises less than 5% by weight, preferably less than 1% by weight, more preferably less than 0.5% by weight, of microfillers, particularly preferably no microfillers, where said microfillers are preferably milled fillers or spherical fillers and have a particle size in the range from 1 to 50 pm and differ in terms of shape and size from the nanosize fillers of component b).

10. The use as claimed in any of claims 1-5, the process as claimed in claim 6 or the shaped dental part as claimed in any of claims 7-9, characterized in that the composite resin composition comprises
less than 0.5% by weight, preferably less than 0.01% by weight, of thixotropy-inducing agents, particularly preferably no thixotropy-inducing agents, and/or
further dental additives, including fluorescent dyes.
